(19) United States
(12) Patent Application Publication
(10) Pub. No.: US 2021/0179673 A1
(43) Pub. Date: Jun. 17, 2021

(54) METHODS AND MATERIALS FOR TREATING CANCER

(71) Applicants: Yon Son Betty Kim, Jacksonville Beach, FL (US); Wen Jiang, Dallas, TX (US)

(72) Inventors: Yon Son Betty Kim, Jacksonville Beach, FL (US); Wen Jiang, Dallas, TX (US)

(21) Appl. No.: 17/263,689

(22) PCT Filed: Jul. 26, 2019
(86) PCT No.: PCT/US2019/043747

§ 371 Date: Jan. 27, 2021

Related U.S. Application Data
(60) Provisional application No. 62/711,380, filed on Jul. 27, 2018.

(51) Int. Cl. C07K 14/36; A61P 35/00; C12N 5/0786

(52) U.S. Cl. CPC C07K 14/36; C07K 2319/33; C12N 5/0645; A61P 35/00

(57) ABSTRACT

This document provides methods and materials involved in treating cancer. For example, compositions containing a fusion polypeptide (or a polypeptide conjugate) having a tumor-targeting moiety and a phagocyte engaging moiety as well as methods and materials for using such compositions to treat cancer are provided.

### CROSS-REFERENCE TO RELATED APPLICATIONS

This application claims priority to U.S. application Ser. No. 62/711,380, filed on Jul. 27, 2018. The disclosure of the prior application is considered part of the disclosure of this application, and is incorporated in its entirety into this application.

### BACKGROUND

### 1. Technical Field

This document relates to methods and materials involved in treating cancer. For example, this document relates to methods and materials involved in using compositions that contain a fusion polypeptide (or polypeptide conjugate) having a tumor-targeting moiety (e.g., an antibody having binding affinity for an antigen expressed by cancer cells within a mammal such as a Herceptin® antibody) and a phagocyte engaging moiety (e.g., a polypeptide sequence that engages a phagocyte within a mammal such as a Signaling Lymphocytic Activation Molecule Family member 7 (SLAMF7) amino acid sequence) to treat cancer.

### 2. Background Information

The innate immune system is integral to the host's defense against foreign pathogens and essential to mediate cellular homeostasis. The first lines of defense are the professional antigen presenting cells (APCs) of the innate immune system, the gatekeepers of the body's immune system. These cells are designed to detect and eradicate infected or diseased cells (via phagocytosis) within the body and process cellular components and proteins and present them as antigens to the adaptive immune system. The innate immune system alone, however, does not confer patient-specific or disease-specific clearance, nor does it have the ability to generate long-lasting immune memory. The memory and immune surveillance programs for the host are mediated by a more specialized and highly specific adaptive immune system involving T-cells.

The recent success of cancer immunotherapy for treatment-refractory metastatic melanoma, non-small cell lung cancer and renal cell carcinoma has provided new hope that similar approaches can be applied to metastatic breast cancer. However, only a small proportion of cancer patients (˜20%) respond to immune checkpoint monotherapy (Jenkins et al., Br. J. Cancer, 118:9-16 (2018)). In addition, multiple studies demonstrated that hematological malignancies (“liquid tumors”) such as leukemia, lymphomas, and multiple myelomas have significantly higher responses rates to certain immunotherapies as compared to solid tumors (Chen et al., Nature, 544:493-497 (2017)).

### SUMMARY

This document provides methods and materials involved in treating cancer. For example, this document provides compositions containing a fusion polypeptide (or a polypeptide conjugate) having a tumor-targeting moiety and a phagocyte engaging moiety as well as methods and materials for using such compositions to treat cancer. A fusion polypeptide (or a polypeptide conjugate) provided herein can include (a) one or more molecules having the ability to bind to a cancer cell (e.g., a cancer cell of a solid tumor such as a human breast cancer cell), (b) an optional linker component, and (c) one or more molecules having the ability to bind to an antigen presenting cell (e.g., a human macrophage).

As described herein, a fusion polypeptide (or a polypeptide conjugate) provided herein can be designed to include a molecule having the ability to bind to a cancer cell such as an anti-cancer antigen antibody (e.g., an anti-human epidermal growth factor receptor 2 (HER2) antibody such as an Herceptin® antibody (trastuzumab)), an optional linker (e.g., a polypeptide-based linker such as an amino acid segment or streptavidin and biotin components), and a molecule having the ability to bind to an APC such as a SLAMF7 amino acid sequence. Such fusion polypeptides (or polypeptide conjugates) can have the ability to facilitate the phagocytosis of cancer cells by APCs within a mammal's body and to activate down-stream adaptive immune responses (e.g., T cell responses) to treat cancer. In addition, the modular design of the fusion polypeptides (or polypeptide conjugates) provided herein (e.g., a two component modular design or three component modular design) can allow clinicians to readily target tumors of different types or tumors of the same type but from different patients to personalize their therapy. The fusion polypeptides (or polypeptide conjugates) provided herein also allow for the production of a universal cancer immunotherapy platform in a simple and economical manner.

SLAMF7 was discovered to be a “pro-phagocytosis” molecule expressed by liquid tumors, but not by solid tumors (Chen et al., Nature, 544:493-497 (2017)). Studies also demonstrated that without the presence of SLAMF7, the innate immune system (first responders of immunity) cannot detect and engulf solid tumors, and thus allows solid tumors to grow and spread (Chen et al., Nature, 544:493-497 (2017)). As described herein, a fusion polypeptide (or a polypeptide conjugate) designed to include a molecule having the ability to bind to a cancer cell (e.g., a cancer cell of a solid tumor) such as an anti-cancer antigen antibody (e.g., an Herceptin® antibody or an anti-EGFR antibody), an optional linker, and a SLAMF7 amino acid sequence can have the ability to convert solid tumors with limited, or no, cancer immunotherapy responsiveness into solid tumors with increased cancer immunotherapy responsiveness. In some cases, a fusion polypeptide (or a polypeptide conjugate) provided herein that contains a SLAMF7 amino acid sequence can be used to increase the cancer immunotherapy responsiveness of a tumor (e.g., a solid tumor) within a mammal (e.g., a human) having cancer. Examples of cancer immunotherapies that can become more effective within a mammal (e.g., a human) having cancer (e.g., a solid tumor) as a result of administration of a fusion polypeptide (or a polypeptide conjugate) provided herein include, without limitation, chimeric antigen receptor (CAR) T cell anti-cancer therapies (e.g., SLAMF7-CAR T cells (Gogishvili et al., Blood, 130(26):2838-2847 (2017)), treatments with molecules that interfere with T cell checkpoints (e.g., anti-CLTA-4 antibodies), treatments with molecules that interfere with the interaction between PD-1 and PD-L1 (e.g., anti-PD-1 antibodies or anti-PD-L1 antibodies), treatments with molecules that interfere with the interaction between CD47 and SIRPα (e.g., anti-CD47 antibodies or anti-SIRPα antibodies or SIRPα binding peptides), and treatments with molecules that stimulate innate immune responses (e.g., stimulators of interferon gene agonists such as MK-1454, Natural Killer cell activators (e.g., anti-SLAMF7 antibody such as Elotuzumab), and pro-phagocytosis initiators or agonists such as anti-CD47 antibodies). In some cases, a fusion polypeptide (or a polypeptide conjugate) provided herein or a composition containing a fusion polypeptide (or a polypeptide conjugate) provided herein can be used in combination with a cancer immunotherapy (e.g., a cancer immunotherapy described herein) to treat cancer. For example, a fusion polypeptide (or a polypeptide conjugate) provided herein can be used in combination with an anti CTLA-4, anti-PD-1, or anti-PD-L1 antibody to treat cancer (e.g., a solid tumor) within a mammal (e.g., a human). In another example, a fusion polypeptide (or a polypeptide conjugate) provided herein can be used in combination with an anti-CD47 antibody or a SIRPα binding polypeptide to treat cancer (e.g., a solid tumor) within a mammal (e.g., a human). In another example, a fusion polypeptide (or a polypeptide conjugate) provided herein can be used in combination with CAR T cell immunotherapy (e.g., SLAMF7 CAR T cells) to treat cancer (e.g., a solid tumor) within a mammal (e.g., a human). In another example, a fusion polypeptide (or a polypeptide conjugate) provided herein can be used in combination with NK cell activators (e.g., anti-SLAMF7 antibody) to treat cancer (e.g., a solid tumor) within a mammal (e.g., a human).

In general, one aspect of this document features a composition comprising (or consisting essentially of or consisting of) a first molecule having the ability to bind to cancer cells and a second molecule having the ability to bind to antigen presenting cells, wherein the first molecule is non-covalently linked to the second molecule, wherein incubation of the cancer cells and the antigen presenting cells in the presence of the composition results in increased phagocytosis of the cancer cells by the antigen presenting cells as compared to the level of phagocytosis of comparable cancer cells by comparable antigen presenting cells in the absence of the composition. The first molecule can comprise a streptavidin sequence, wherein the second molecule comprises a biotin sequence, and wherein the first molecule is non-covalently linked to the second molecule via a non-covalent linkage between the streptavidin sequence and the biotin sequence. The second molecule can comprise a streptavidin sequence, wherein the first molecule comprises a biotin sequence, and wherein the first molecule is non-covalently linked to the second molecule via a non-covalent linkage between the streptavidin sequence and the biotin sequence. The molecule having the ability to bind to cancer cells can be an anti-cancer antigen antibody. The anti-cancer antigen antibody can be an anti-CD340 antibody, an anti-EGFR antibody, or an anti-PSMA antibody. The molecule having the ability to bind to antigen presenting cells can be a polypeptide. The polypeptide can comprise (or consist essentially of or consist of) a SLAMF7 amino acid sequence. The SLAMF7 amino acid sequence can comprise (or consist essentially of or consist of) the sequence set forth in SEQ ID NO:5. The cancer cells can be breast cancer cells, brain cancer cells, prostate cancer cells, lung cancer cells, or colorectal cancer cells. The cancer cells can be human breast cancer cells, human brain cancer cells, human prostate cancer cells, human lung cancer cells, or human colorectal cancer cells. The antigen presenting cells can be macrophages. The antigen presenting cells can be human macrophages. The increased phagocytosis can be at least a two-fold increase. The increased phagocytosis can be at least a four-fold increase.

In another aspect, this document features a fusion polypeptide comprising (or consisting essentially of or consisting of) a first amino acid segment having the ability to bind to cancer cells and a second amino acid segment having the ability to bind to antigen presenting cells, wherein incubation of the cancer cells and the antigen presenting cells in the presence of the fusion polypeptide results in increased phagocytosis of the cancer cells by the antigen presenting cells as compared to the level of phagocytosis of comparable cancer cells by comparable antigen presenting cells in the absence of the fusion polypeptide. The first amino acid segment can be linked to the second amino acid segment via a linker amino acid sequence. The first amino acid segment having the ability to bind to cancer cells can be an anti-cancer antigen antibody. The anti-cancer antigen antibody can be an anti-CD340 antibody, an anti-EGFR antibody, or an anti-PSMA antibody. The second amino acid segment can comprise (or consist essentially of or consist of) a SLAMF7 amino acid sequence. The SLAMF7 amino acid sequence can comprise (or consist essentially of or consist of) the amino acid sequence set forth in SEQ ID NO:5. The cancer cells can be breast cancer cells, brain cancer cells, prostate cancer cells, lung cancer cells, or colorectal cancer cells. The cancer cells can be human breast cancer cells, human brain cancer cells, human prostate cancer cells, human lung cancer cells, or human colorectal cancer cells. The antigen presenting cells can be macrophages. The antigen presenting cells can be human macrophages. The increased phagocytosis can be at least a two-fold increase. The increased phagocytosis can be at least a four-fold increase.

In another aspect, this document features a method for treating a mammal having cancer. The method comprises (or consists essentially of or consists of) administering to the mammal a composition. The composition can comprise (or consist essentially of or consist of) a first molecule having the ability to bind to cancer cells and a second molecule having the ability to bind to antigen presenting cells, wherein the first molecule is non-covalently linked to the second molecule, wherein incubation of the cancer cells and the antigen presenting cells in the presence of the composition results in increased phagocytosis of the cancer cells by the antigen presenting cells as compared to the level of phagocytosis of comparable cancer cells by comparable antigen presenting cells in the absence of the composition. The first molecule can comprise a streptavidin sequence, wherein the second molecule comprises a biotin sequence, and wherein the first molecule is non-covalently linked to the second molecule via a non-covalent linkage between the streptavidin sequence and the biotin sequence. The second molecule can comprise a streptavidin sequence, wherein the first molecule comprises a biotin sequence, and wherein the first molecule is non-covalently linked to the second molecule via a non-covalent linkage between the streptavidin sequence and the biotin sequence. The molecule having the ability to bind to cancer cells can be an anti-cancer antigen antibody. The anti-cancer antigen antibody can be an anti-CD340 antibody, an anti-EGFR antibody, or an anti-PSMA antibody. The molecule having the ability to bind to antigen presenting cells can be a polypeptide. The polypeptide can comprise (or consist essentially of or consist of) a SLAMF7 amino acid sequence. The SLAMF7 amino acid sequence can comprise (or consist essentially of or consist of) the sequence set forth in SEQ ID NO:5. The cancer cells can be breast cancer cells, brain cancer cells, prostate cancer cells, lung cancer cells, or colorectal cancer cells. The cancer cells can be human breast cancer cells, human brain cancer cells, human prostate cancer cells, human lung cancer cells, or human colorectal cancer cells. The antigen presenting cells can be macrophages. The antigen presenting cells can be human macrophages. The increased phagocytosis can be at least a two-fold increase. The increased phagocytosis can be at least a four-fold increase. The mammal can be a human. The cancer can be breast cancer, brain cancer, prostate cancer, lung cancer, or colorectal cancer. The composition can be administered by injection, ingestion, or inhalation.

In another aspect, this document features a method for treating a mammal having cancer. The method comprises (or consists essentially of or consists of) administering to the mammal a fusion polypeptide. The fusion polypeptide can comprise (or consist essentially of or consist of) a first amino acid segment having the ability to bind to cancer cells and a second amino acid segment having the ability to bind to antigen presenting cells, wherein incubation of the cancer cells and the antigen presenting cells in the presence of the fusion polypeptide results in increased phagocytosis of the cancer cells by the antigen presenting cells as compared to the level of phagocytosis of comparable cancer cells by comparable antigen presenting cells in the absence of the fusion polypeptide. The first amino acid segment can be linked to the second amino acid segment via a linker amino acid sequence. The first amino acid segment having the ability to bind to cancer cells can be an anti-cancer antigen antibody. The anti-cancer antigen antibody can be an anti-CD340 antibody, an anti-EGFR antibody, or an anti-PSMA antibody. The second amino acid segment can comprise (or consist essentially of or consist of) a SLAMF7 amino acid sequence. The SLAMF7 amino acid sequence can comprise (or consist essentially of or consist of) the amino acid sequence set forth in SEQ ID NO:5. The cancer cells can be breast cancer cells, brain cancer cells, prostate cancer cells, lung cancer cells, or colorectal cancer cells. The cancer cells can be human breast cancer cells, human brain cancer cells, human prostate cancer cells, human lung cancer cells, or human colorectal cancer cells. The antigen presenting cells can be macrophages. The antigen presenting cells can be human macrophages. The increased phagocytosis can be at least a two-fold increase. The increased phagocytosis can be at least a four-fold increase. The mammal can be a human. The cancer can be breast cancer, brain cancer, prostate cancer, lung cancer, or colorectal cancer. The composition can be administered by injection, ingestion, or inhalation.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of an example of a fusion polypeptide (right) and a conjugate (left) according to some embodiments. The configuration on the left contains optional streptavidin and biotin linkers for form a conjugate, and the configuration on the right uses an optional amino acid linker sequence within the fusion polypeptide. The top component of each is a phagocyte engaging moiety (e.g., a SLAMF7 amino acid sequence), and the bottom component of each is a tumor-targeting moiety (e.g., an antibody sequence that having binding affinity for a cancer antigen).

FIG. 2 (top) is a schematic of a conjugate according to some embodiments. In this example, the conjugate contains a SLAMF7 amino acid sequence attached to a streptavidin sequence and a Herceptin antibody sequence attached to a biotin sequence. This conjugated can be designated as S-H. The interaction between streptavidin and biotin is used to form the conjugate. FIG. 2 (bottom) is a schematic of one mechanism of action for the conjugate. Briefly, the conjugate can target tumor-specific antigens (such as Her2). Once engaged, the SLAMF7 sequence can trigger the innate immune system (e.g., macrophages) to internalize cancer cells via the process of phagocytosis. Once the tumor cells are internalized, fragments of the tumor polypeptides can be presented on the surface of the macrophages via the process of antigen presentation. Presentation of tumor-specific polypeptides can illicit T cells to be primed and/or activated to kill and eradicate tumor cells. A portion of T cells can become memory T cells, resulting in long-term anti-tumor immunity.

FIG. 3. Expression of SLAMF7 on solid tumors. Unlike hematological (blood-born tumors, also known as “liquid tumors”), SLAMF7 expression is low in solid tumors. Eight different cancer cell lines were tested to determine expression levels of SLAMF7.

FIG. 4. Affinity assay and tumor cell targeting capability studies of S-H conjugate of FIG. 2. These results demonstrate tumor specific targeting ability of S-H with very low non-specific targeting of the S-H conjugate. Compared to cells that do not over express Her2 (E0771), robust targeting was demonstrate in cells with overexpression of Her2 (E0771/E2).

FIG. 5. Tumor cell clearance/phagocytosis assay. This study demonstrates that the tumor specific targeting ability of the S-H conjugate resulted in increased clearance of tumor cells by macrophages. Compared with cells that do not over express Her2 (E0771), tumor cell phagocytosis rates doubled in cells targeted with overexpression of Her2 (E0771/E2).

FIG. 6. Antigen presentation of tumor polypeptide studies. This study demonstrates that the tumor-specific targeting ability of the S-H conjugate not only resulted in increased clearance of tumor cells by macrophages. It also resulted in increased cross presentation of antigens (e.g., polypeptides derived from cytoplasmic ovalbumin) by phagocytes treated with the SLAMF7-Herceptin (S-H) conjugate as compared to the level observed when Herceptin and recombinant SLAMF7 protein were given together without conjugation.

FIG. 7. Antigen presentation of tumor polypeptide studies. This study uses immunohistochemistry to demonstrate that the tumor specific targeting ability not only resulted in increased clearance of tumor cells by macrophages, but also resulted in cross presentation of antigens were observed. Antigen cross presentation by macrophages is shown using a labeled antibody that specifically recognizes MHC molecules bound with a cOVA derived antigenic peptide. The top right panel exhibits more staining with the labelled antibody as compared to the other panels.

FIG. 8. T cell activation. The SLAMF7-Herceptin (S-H) conjugate results in improved T cell activation as compared to when SLAMF7 or Herceptin were given together but without conjugation. The generation of memory T cells and antigen specific T cell further confirmed that the response was tumor specific.

FIG. 9 contains the amino acid sequence for a mouse SLAMF7 polypeptide.

FIG. 10 contains the amino acid sequence for a Herceptin antibody.

FIG. 11 lists experimental groups for breast cancer model.

FIG. 12 lists experimental groups for brain tumor model.

### DETAILED DESCRIPTION

This document provides methods and materials involved in treating cancer. For example, this document provides compositions containing a fusion polypeptide (or a polypeptide conjugate) having a tumor-targeting moiety and a phagocyte engaging moiety as well as methods and materials for using such compositions to treat cancer. A fusion polypeptide (or a polypeptide conjugate) provided herein can include (a) one or more molecules having the ability to bind to a cancer cell (e.g., a cancer cell of a solid tumor such as a human breast cancer cell), (b) an optional linker component, and (c) one or more molecules having the ability to bind to an antigen presenting cell (e.g., a human macrophage). An example of a molecule having the ability to bind to an antigen presenting cell (e.g., a human macrophage) includes, without limitation, a SLAMF7 polypeptide.

As described herein, cancer cells (e.g., cancer cells of a solid tumor) can be “artificially” tagged with a SLAMF7 polypeptide so that the body can sense these tumors as being “liquid tumors” and effectively clear them from the body. In some cases, cancer cells (e.g., cancer cells of a solid tumor) can be converted into blood cancers in the eyes of the host's immune system in such a way that immunotherapies can work more efficaciously against those cancer cells. In some cases, a modular fusion polypeptide (or conjugate) described herein can be designed to be bispecific with two or three components: (1) an antibody ligand that targets specific tumor antigens, (2) an optional linker, and (3) a SLAMF7 amino acid sequence. When fully assembled and placed in contact with cancer cells that the fusion polypeptide (or conjugate) was designed to target, the fusion polypeptide (or conjugate) can target those cells (e.g., solid tumor cells), which can then be seen as presenting SLAMF7 amino acid sequences on their surface. In the eyes of the host's immune system, these modified cancer cells can possess characteristics similar to those of a hematologic cancer. In some cases, cancer cells modified with a fusion polypeptide (or conjugate) provided herein can exhibit significantly improved responses to immunotherapy drugs, which, in the case of solid tumor cells, they would otherwise be resistant to in the absence of the modification with the SLAMF7-containing fusion polypeptide (or conjugate).

A fusion polypeptide (or conjugate) provided herein can be readily modified to target different solid tumors, and potentially prevent distant spread of disease (metastasis) and produce a long-term durable immunity. In some cases, a fusion polypeptide (or conjugate) provided herein can provide a strategy to immunologically convert solid cancers in a way that renders non-responders of immunotherapy into responders.

The methods and materials provided herein can be used to treat any appropriate type of cancer. For example, the methods and materials provided herein can be used to treat breast cancer, brain cancer, prostate cancer, lung cancer, colorectal cancer, skin cancer, head and neck cancer, or pancreatic cancer

In some cases, the methods and materials provided herein can be used to treat cancer (e.g., breast cancer) in any appropriate type of mammal including, without limitation, mice, rats, dogs, cats, horses, cows, pigs, monkeys, and humans.

In general, a fusion polypeptide (or a polypeptide conjugate) provided herein can be designed to include a molecule having the ability to bind to a cancer cell and a molecule having the ability to bind to an APC. Examples of molecules having the ability to bind to a cancer cell that can be used to make a fusion polypeptide (or a polypeptide conjugate) provided herein include, without limitation, antibodies such as anti-cancer antigen antibodies, non-antibody polypeptides, antibody fragments, and recombinant proteins. Examples of anti-cancer antigen antibodies having the ability to bind to a cancer cell that can be used to make a fusion polypeptide (or a polypeptide conjugate) provided herein include, without limitation, anti-CD340 antibodies (e.g., anti-human epidermal growth factor receptor 2 (HER2) antibodies such as Herceptin®), anti-EGFR antibodies (e.g., anti-human epidermal growth factor receptor antibodies), anti-EGFRviii antibodies (e.g., anti-human epidermal growth factor receptor variant 3 antibodies), anti-PSMA antibodies (e.g., anti-human prostate specific membrane antigen antibodies), anti-CEA antibodies (e.g., anti-human carcinoembryonic antigen antibodies), anti-CA125 antibodies (e.g., anti-human cancer antigen 125 antibodies), anti-CD20 antibodies (e.g., anti-human cluster antigen 20 antibodies), anti-CD30 antibodies, anti-CD33 antibodies, and anti-GD antibodies (anti-Gangliosides antibodies). In some cases, single-chain antibodies, antibody fragments, nanobodies, full antibodies, or polypeptides can be used. Examples of non-antibody polypeptides having the ability to bind to a cancer cell that can be used to make a fusion polypeptide (or a polypeptide conjugate) provided herein include, without limitation, RGD polypeptides, EGFR-specific polypeptides, and HER2 polypeptides.

Examples of molecules having the ability to bind to an APC that can be used to make a fusion polypeptide (or a polypeptide conjugate) provided herein include, without limitation, SLAMF7 polypeptides. A SLAMF7 polypeptide used to make a fusion polypeptide (or a polypeptide conjugate) provided herein can have the amino acid sequence set forth in SEQ ID NO:5. Other examples of SLAMF7 polypeptides that can be used to make a fusion polypeptide (or a polypeptide conjugate) provided herein include, without limitation, the SLAMF7 polypeptide set forth in GenBank

Accession No. Q9NQ25 and the SLAMF7 polypeptide set forth in GenBank Accession No. Q8BHK6. In some cases, a portion of a full-length SLAMF7 polypeptide can be used to make a fusion polypeptide (or a polypeptide conjugate) provided herein. For example, any of the SLAMF7 amino acid sequences set forth in Table 1 can be used to make a fusion polypeptide (or a polypeptide conjugate) provided herein. A fusion polypeptide (or polypeptide conjugate) provided herein can include a segment of full length human SLAMF7 (e.g., a SLAMF7 extracellular domain) as shown in Table 1.

TABLE 1
Human and mouse SLAMF7 amino acid sequences.

| Name | | |
|---|---|---|
| ID | | |
| NO: | Sequence | SEQ |
| hSLAMF7₁₋₃₃₅ | MAGSPTCLTLIYILWQLTGSAASGPVKELVGSVGGAV | 3 |
| (full length) | TFPLKSKVKQVDSIVWTFNTTPLVTIQPEGGTIIVTQN | |
| | RNRERVDFPDGGYSLKLSKLKKNDSGIYYVGIYSSSL | |
| | QQPSTQEYVLHVYEHLSKPKVTMGLQSNKNGTCVTN | |
| | LTCCMEHGEEDVIYTWKALGQAANESHNGSILPISWR | |
| | WGESDMTFICVARNPVSRNFSSPILARKLCEGAADDP | |
| | DSSMVLLCLLLVPLLLSLFVLGLFLWFLKRERQEEYIE | |
| | EKKRVDICRETPNICPHSGENTEYDTIPHTNRTILKEDP | |
| | ANTVYSTVEIPKKMENPHSLLTMPDTPRLFAYENVI | |
| hSLAMF7₂₃₋₃₃₅ | SGPVKELVGSVGGAVTFPLKSKVKQVDSIVWTFNTTP | 4 |
| (mature | LVTIQPEGGTIIVTQNRNRERVDFPDGGYSLKLSKLKK | |
| polypeptide) | NDSGIYYVGIYSSSLQQPSTQEYVLHVYEHLSKPKVT | |
| | MGLQSNKNGTCVTNLTCCMEHGEEDVIYTWKALGQ | |
| | AANESHNGSILPISWRWGESDMTFICVARNPVSRNFS | |
| | SPILARKLCEGAADDPDSSMVLLCLLLVPLLLSLFVLG | |
| | LFLWFLKRERQEEYIEEKKRVDICRETPNICPHSGENT | |
| | EYDTIPHTNRTILKEDPANTVYSTVEIPKKMENPHSLL | |
| | TMPDTPRLFAYENVI | |
| hSLAMF7₂₃₋₂₂₆ | SGPVKELVGSVGGAVTFPLKSKVKQVDSIVWTFNTTP | 5 |
| (extracellular | LVTIQPEGGTIIVTQNRNRERVDFPDGGYSLKLSKLKK | |
| domain) | NDSGIYYVGIYSSSLQQPSTQEYVLHVYEHLSKPKVT | |
| | MGLQSNKNGTCVTNLTCCMEHGEEDVIYTWKALGQ | |
| | AANESHNGSILPISWRWGESDMTFICVARNPVSRNFS | |
| | SPILARKLCEGAADDPDSSM | |
| hSLAMF7₂₃₋₁₂₄ | SGPVKELVGSVGGAVTFPLKSKVKQVDSIVWTFNTTP | 6 |
| (Ig-like V-type | LVTIQPEGGTIIVTQNRNRERVDFPDGGYSLKLSKLKK | |
| domain) | NDSGIYYVGIYSSSLQQPSTQEYVLHV | |
| hSLAMF7₁₃₁₋₂₀₆ | PKVTMGLQSNKNGTCVTNLTCCMEHGEEDVIYTWK | 7 |
| (Ig-like C2-type) | ALGQAANESHNGSILPISWRWGESDMTFICVARNPVS | |
| | RNFS | |
| hSLAMF7₃₁₋₁₂₅ | GSVGGAVTFPLKSKVKQVDSIVWTFNTTPLVTIQPEG | 8 |
| (immunoglobulin- | GTIIVTQNRNRERVDFPDGGYSLKLSKLKKNDSGIYY | |
| like domain) | VGIYSSSLQQPSTQEYVLHVY | |
| hSLAMF7₂₇₋₁₀₄ | KELVGSVGGAVTFPLKSKVKQVDSIVWTFNTTPLVTI | 9 |
| (immunoglobulin | QPEGGTIIVTQNRNRERVDFPDGGYSLKLSKLKKNDS | |
| like domain) | GIYY | |
| mSLAMF₇₁₋₃₃₃ | MARFSTYIIFTSVLCQLTVTAASGTLKKVAGALDGSV | 10 |
| (full length) | TFTLNITEIKVDYVVWTFNTFFLAMVKKDGVTSQSSN | |
| | KERIVFPDGLYSMKLSQLKKNDSGAYRAEIYSTSSQA | |
| | SLIQEYVLHVYKHLSRPKVTIDRQSNKNGTCVINLTC | |
| | STDQDGENVTYSWKAVGQGDNQFHDGATLSIAWRS | |
| | GEKDQALTCMARNPVSNSFSTPVFPQKLCEDAATDL | |
| | TSLRGILYILCFSAVLILFAVLLTIFHTTWIKKGKGCEE | |
| | DKKRVDRHQEMPDLCPHLEENADYDTIPYTEKRRPE | |
| | EDAPNTFYSTVQIPKVVKSPSSLPAKPLVPRSLSFENVI | |
| mSLAMF7₂₃₋₃₃₃ | SGTLKKVAGALDGSVTFTLNITEIKVDYVVWTFNTFF | 11 |
| (mature | LAMVKKDGVTSQSSNKERIVFPDGLYSMKLSQLKKN | |
| polypeptide) | DSGAYRAEIYSTSSQASLIQEYVLHVYKHLSRPKVTID | |
| | RQSNKNGTCVINLTCSTDQDGENVTYSWKAVGQGD | |
| | NQFHDGATLSIAWRSGEKDQALTCMARNPVSNSFST | |
| | PVFPQKLCEDAATDLTSLRGILYILCFSAVLILFAVLLT | |
| | IFHTTWIKKGKGCEEDKKRVDRHQEMPDLCPHLEEN | |
| | ADYDTIPYTEKRRPEEDAPNTFYSTVQIPKVVKSPSSL | |
| | PAKPLVPRSLSFENVI | |
| mSLAMF7₂₃₋₂₂₄ | SGTLKKVAGALDGSVTFTLNITEIKVDYVVWTFNTFF | 1 |
| (extracellular | LAMVKKDGVTSQSSNKERIVFPDGLYSMKLSQLKKN | |
| domain) | DSGAYRAEIYSTSSQASLIQEYVLHVYKHLSRPKVTID | |
| | RQSNKNGTCVINLTCSTDQDGENVTYSWKAVGQGD | |
| | NQFHDGATLSIAWRSGEKDQALTCMARNPVSNSFST | |
| | PVFPQKLCEDAATDLTSLRG | |
| mSLAMF7₂₇₋₁₁₂ | KKVAGALDGSVTFTLNITEIKVDYVVWTFNTFFLAM | 12 |
| (Ig-like V-type | VKKDGVTSQSSNKERIVFPDGLYSMKLSQLKKNDSG | |
| domain) | AYRAEIYSTSSQAS | |
| mSLAMF7₁₂₈₋₂₀₃ | PKVTIDRQSNKNGTCVINLTCSTDQDGENVTYSWKA | 13 |
| (Ig-like C2-type) | VGQGDNQFHDGATLSIAWRSGEKDQALTCMARNPV | |
| | SNSFS | |
| mSLAMF7₃₁₋₁₂₂ | GALDGSVTFTLNITEIKVDYVVWTFNTFFLAMVKKD | 14 |
| (immunoglobulin | GVTSQSSNKERIVFPDGLYSMKLSQLKKNDSGAYRA | |
| domain) | EIYSTSSQASLIQEYVLHVY | |
| mSLAMF7₁₂₇₋₁₉₆ | RPKVTIDRQSNKNGTCVINLTCSTDQDGENVTYSWK | 15 |
| (immunoglobulin | AVGQGDNQFHDGATLSIAWRSGEKDQALTCMARN | |
| domain) | | |

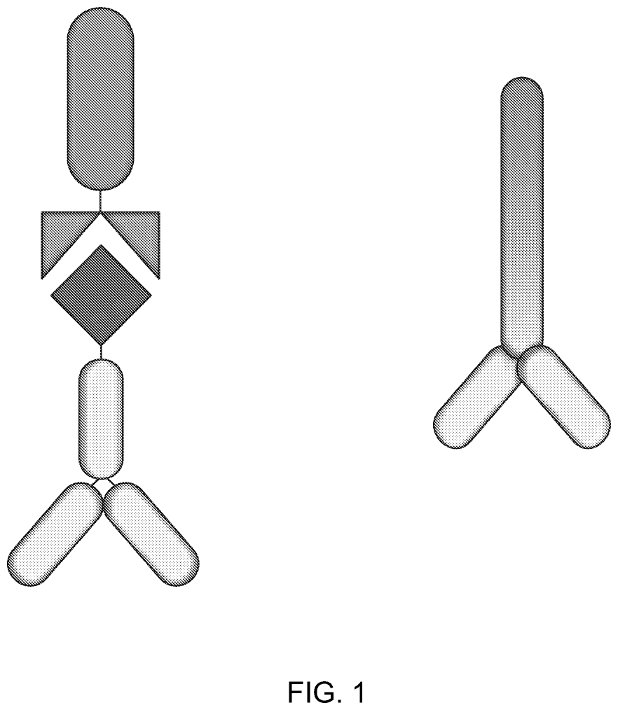

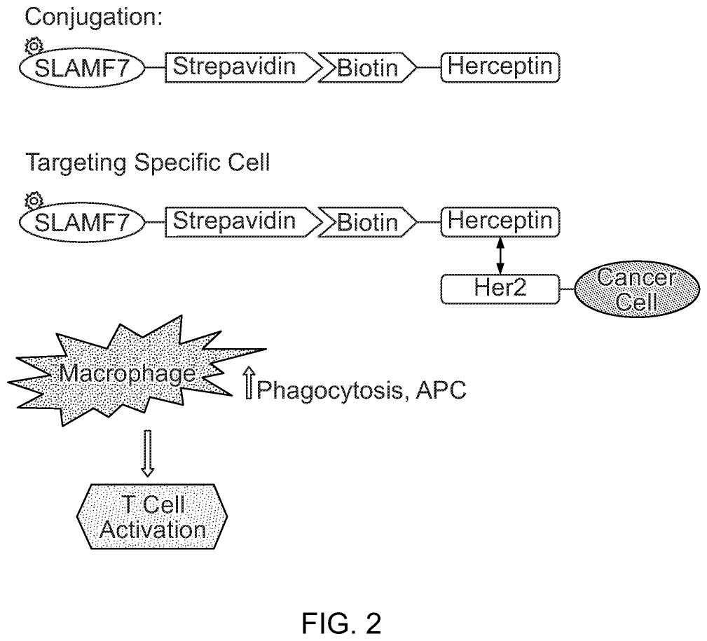

In some cases, a fusion polypeptide (or a polypeptide conjugate) provided herein can be designed as shown in FIG. 1 or FIG. 2. For example, in some cases, a fusion polypeptide (or a polypeptide conjugate) provided herein can be entirely composed of polypeptides or amino acids. In some cases, a fusion polypeptide provided herein can be a fusion protein composed of a single amino acid chain, and a conjugate provided herein can be a conjugate of different polypeptides that are non-covalently linked together. In some cases, streptavidin and biotin can be used to link the different polypeptide components together. Other examples of linkers that can be used to link (a) one or more molecules having the ability to bind to a cancer cell (e.g., a human breast cancer cell) and (b) one or more molecules having the ability to bind to an APC (e.g., a human macrophage) such as a SLAMF7 amino acid sequence together include, without limitation, chemical linkers such as acylhydrazones and other polypeptide linkers (synthetic or biological). In another example, nanoparticle can be used as a linker. For example, both (a) a cancer-binding molecule and (b) an APC-binding molecule (e.g., SLAMF7 polypeptide) can be conjugated to and connected by a nanoparticle linker.

In some cases, a fusion polypeptide (or a polypeptide conjugate) provided herein can be designed to include a therapeutic agent, a label, and/or a contrast agent. For example, a fusion polypeptide (or a polypeptide conjugate) provided herein can be designed to include one, two, three, four, five, six, or more therapeutic agents. Examples of therapeutic agents that can be incorporated into a fusion polypeptide (or a polypeptide conjugate) provided herein include, without limitation, doxorubicin, cisplatin, carboplatin, temozolomide, docetaxel, and 5-FU. In some cases, a fusion polypeptide (or a polypeptide conjugate) provided herein can be designed to include one, two, three, four, five, six, or more contrast agent materials. Examples of labels that can be incorporated into a fusion polypeptide (or a polypeptide conjugate) provided herein include, without limitation, radioactive tracers or labels such as carbon-14 (¹⁴C), hydrogen isotopes (¹H, ²H, ³H), and ¹³N, polypeptide tags such as green fluorescent protein (GFP), and epitope tags such as FLAG, poly-His, and glutathione-S-transferase (GST). Examples of contrast agent materials that can be incorporated into a fusion polypeptide (or a polypeptide conjugate) provided herein include, without limitation, gadolinium, iodide, iron oxide, FDG, radio-isotopes, and organic dyes.

In some cases, a fusion polypeptide (or a polypeptide conjugate) provided herein can have the ability to facilitate the phagocytosis of cancer cells by APCs within a mammal's body and to activate down-stream adaptive immune responses (e.g., T cell responses) to treat cancer. For example, a fusion polypeptide (or a polypeptide conjugate) provided herein can be designed to recognize simultaneously cancer cells (which can deliver the fusion polypeptide (or polypeptide conjugate) provided herein to the area of interest, can limit non-specific immune reactions, and can spare normal surrounding tissues, thereby minimizing adverse effects) and APCs (which can enable these cells to initiate the first stages of a cancer cell clearance process). The ability to home and heighten the localization of APC's to the tumor microenvironment can increase tumor phagocytosis (e.g., tumor cell internalization). Once internalized, APCs can present fragments of the tumor antigens to the membrane surface via MHC molecules on the cell surface. This, in turn, can enhance T cell recognition of the cancer cells within a mammal.

As described herein, a fusion polypeptide (or a polypeptide conjugate) provided herein (or a composition containing a fusion polypeptide or a polypeptide conjugate) can be administered to a mammal to treat cancer. Any appropriate method can be used to administer a fusion polypeptide (or a polypeptide conjugate) provided herein (or a composition containing a fusion polypeptide or a polypeptide conjugate provided herein) to a mammal. For example, a fusion polypeptide (or a polypeptide conjugate) provided herein (or a composition containing a fusion polypeptide or a polypeptide conjugate provided herein) can be administered via injection (e.g., subcutaneous injection, intramuscular injection, intravenous injection, or intrathecal injection).

Before administering a fusion polypeptide (or a polypeptide conjugate) provided herein (or a composition containing a fusion polypeptide or a polypeptide conjugate provided herein) to a mammal, the mammal can be assessed to determine whether or not the mammal has cancer (e.g., breast cancer). Any appropriate method can be used to determine whether or not a mammal has cancer. For example, a mammal (e.g., human) can be identified as having cancer using standard diagnostic techniques. In some cases, a tissue biopsy can be collected and analyzed to determine whether or not a mammal has cancer.

After identifying a mammal as having cancer (e.g., breast cancer), the mammal can be administered a fusion polypeptide (or a polypeptide conjugate) provided herein (or a composition containing a fusion polypeptide or a polypeptide conjugate provided herein). For example, a fusion polypeptide (or a polypeptide conjugate) provided herein (or a composition containing a fusion polypeptide or a polypeptide conjugate provided herein) can be administered prior to or in lieu of surgical resection of a tumor. In some cases, a fusion polypeptide (or a polypeptide conjugate) provided herein (or a composition containing a fusion polypeptide or a polypeptide conjugate provided herein) can be administered following resection of a tumor.

A fusion polypeptide (or a polypeptide conjugate) provided herein (or a composition containing a fusion polypeptide or a polypeptide conjugate provided herein) can be administered to a mammal in any appropriate amount, at any appropriate frequency, and for any appropriate duration effective to achieve a desired outcome (e.g., to increase progression-free survival). In some cases, a fusion polypeptide (or a polypeptide conjugate) provided herein (or a composition containing a fusion polypeptide or a polypeptide conjugate provided herein) can be administered to a mammal having cancer (e.g., breast cancer) to reduce the progression rate of the cancer by 5, 10, 25, 50, 75, 100, or more percent. For example, the progression rate can be reduced such that no additional cancer progression is detected. Any appropriate method can be used to determine whether or not the progression rate of cancer is reduced. For example, the progression rate of cancer can be assessed by imaging tissue at different time points and determining the amount of cancer cells present. The amounts of cancer cells determined within tissue at different times can be compared to determine the progression rate. After treatment as described herein, the progression rate can be determined again over another time interval. In some cases, the stage of cancer after treatment can be determined and compared to the stage before treatment to determine whether or not the progression rate was reduced.

In some cases, a fusion polypeptide (or a polypeptide conjugate) provided herein (or a composition containing a fusion polypeptide or a polypeptide conjugate provided herein) can be administered to a mammal having cancer (e.g., breast cancer) in combination with a cancer immunotherapy (e.g., anti-CD47 antibodies, anti-SIRPα antibodies, NK cell activators, CAR T cells, anti-CTLA-4 antibodies, anti-PD-1 antibodies, or anti-PD-L1 antibodies) under conditions where progression-free survival is increased (e.g., by 5, 10, 25, 50, 75, 100, or more percent) as compared to the median progression-free survival of corresponding mammals having untreated cancer (e.g., untreated breast cancer). Progression-free survival can be measured over any appropriate length of time (e.g., one month, two months, three months, four months, five months, six months, or longer). For combination therapies, a fusion polypeptide or polypeptide conjugate provided herein can be administered concurrently or sequentially with another therapy. In one example, a fusion polypeptide or polypeptide conjugate provided herein can be administered prior to administration of CART cells (e.g., SLAMF7-CAR T cells (Gogishvili et al., Blood, 130(26):2838-2847 (2017)). In another example, a fusion polypeptide or polypeptide conjugate provided herein can be administered prior to administration of an NK cell activator biologic drug (e.g., an anti-SLAMF7 antibody).

An effective amount of a fusion polypeptide (or a polypeptide conjugate) provided herein (or a composition containing a fusion polypeptide or a polypeptide conjugate provided herein) can be any amount that reduces the progression rate of cancer (e.g., breast cancer), increases the progression-free survival rate, increases the median time to progression, or increases the effectiveness of a cancer immunotherapy without producing significant toxicity to the mammal. Typically, an effective amount of a fusion polypeptide (or a polypeptide conjugate) provided herein (or a composition containing a fusion polypeptide or a polypeptide conjugate provided herein) can be from about 10 mg/m² to about 200 mg/m². If a particular mammal fails to respond to a particular amount, then the amount a fusion polypeptide (or a polypeptide conjugate) provided herein (or a composition containing a fusion polypeptide or a polypeptide conjugate provided herein) can be increased by, for example, two fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the cancer (e.g., breast cancer) may require an increase or decrease in the actual effective amount administered.

The frequency of administration of a fusion polypeptide (or a polypeptide conjugate) provided herein (or a composition containing a fusion polypeptide or a polypeptide conjugate provided herein) can be any frequency that reduces the progression rate of cancer (e.g., skin cancer), increases the progression-free survival rate, increases the median time to progression, or increases the effectiveness of a cancer immunotherapy without producing significant toxicity to the mammal. For example, the frequency of administration can be from about once a month to about three times a month, or from about twice a month to about six times a month, or from about once every two months to about three times every two months. The frequency of administration can remain constant or can be variable during the duration of treatment. A course of treatment with a fusion polypeptide (or a polypeptide conjugate) provided herein (or a composition containing a fusion polypeptide or a polypeptide conjugate provided herein) can include rest periods. For example, a fusion polypeptide (or a polypeptide conjugate) provided herein (or a composition containing a fusion polypeptide or a polypeptide conjugate provided herein) can be administered over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the cancer may require an increase or decrease in administration frequency.

An effective duration for administering a fusion polypeptide (or a polypeptide conjugate) provided herein (or a composition containing a fusion polypeptide or a polypeptide conjugate provided herein) can be any duration that reduces the progression rate of cancer (e.g., breast cancer), increases the progression-free survival rate, increases the median time to progression, or increases the effectiveness of a cancer immunotherapy without producing significant toxicity to the mammal. Thus, the effective duration can vary from several days to several weeks, months, or years. In general, the effective duration for the treatment of cancer can range in duration from several weeks to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the cancer (e.g., breast cancer).

A composition containing a fusion polypeptide or a polypeptide conjugate provided herein can be in any appropriate form. For example, a composition containing a fusion polypeptide or a polypeptide conjugate provided herein can be in the form of a solution or powder with or without a diluent to make an injectable suspension. A composition containing a fusion polypeptide or a polypeptide conjugate provided herein also can contain additional ingredients including, without limitation, pharmaceutically acceptable vehicles. A pharmaceutically acceptable vehicle can be, for example, saline, water, lactic acid, mannitol, or combinations thereof.

After administering a fusion polypeptide (or a polypeptide conjugate) provided herein (or a composition containing a fusion polypeptide or a polypeptide conjugate provided herein) to a mammal, the mammal can be monitored to determine whether or not the cancer (e.g., breast cancer) was treated. For example, a mammal can be assessed after treatment to determine whether or not the progression rate of cancer was reduced (e.g., stopped) or to determine whether or not the effectiveness of a cancer immunotherapy was increased. As described herein, any appropriate method can be used to assess progression, survival rates, and cancer immunotherapy effectiveness.

The invention will be further described in the following examples, which do not limit the scope of the invention described in the claims.

### EXAMPLES

### Example 1

### Tagging Cancer Cells with a Phagocyte Engaging Moiety to Treat Cancer within a Mammal

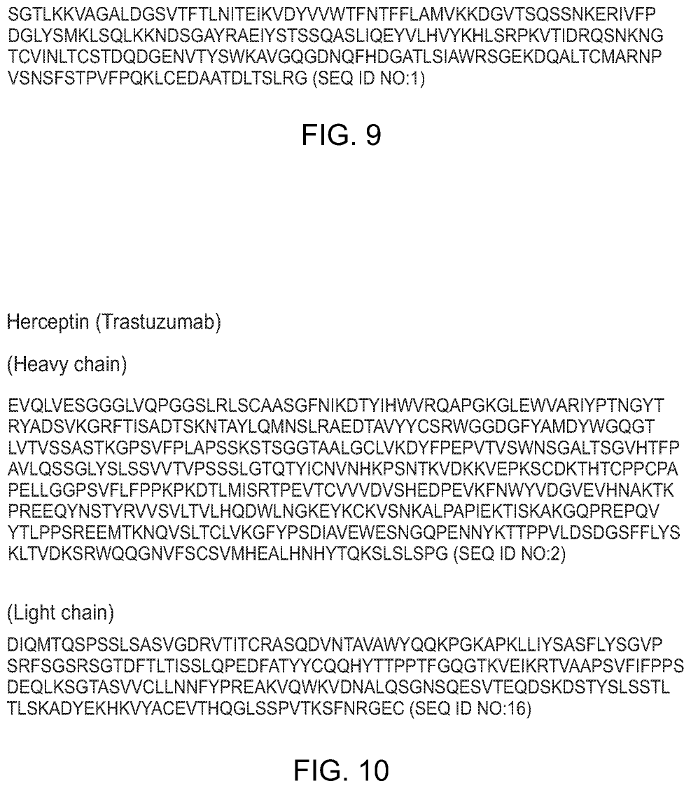

A SLAMF7 amino acid sequence was conjugated with purified monomeric streptavidin to form SLAMF7-streptavidin, and trastuzumab (Herceptin®), a monoclonal antibody that binds specifically to HER2, was conjugated with recombinant biotin via covalent linkage to form Herceptin-biotin. The SLAMF7 amino acid sequence is set forth in FIG. 9, and the amino acid sequence of the Herceptin antibody is set forth in FIG. 10. SLAMF7-streptavidin and Herceptin-biotin were linked together via direct binding between streptavidin-biotin to form a conjugate referred to herein as S-H (FIG. 2). Briefly, SLAMF7 was labeled with Alexa fluor dye 647 (Invitrogen Alexa fluor 647 antibody labeling kit), and a 1 M sodium bicarbonate solution was made by resuspending Component B in 1 mL of deionized water. The antibody (SLAMF7) concentration was adjusted to 1.0 mg/mL. Then, 1/10th volume of the 1 M sodium bicarbonate solution was added, and 100 μL of the antibody was added to the vial of Alexa Fluor dye. The mixture was inverted to mix and incubate for 1 hour at room temperature.

To assemble a purification column, 1.5 mL of a resin bed was prepared and the column was centrifuged at 1100×g for 3 minutes. The reaction mixture was added to the column, which was centrifuged at 1100×g for 5 minutes to collect the labeled antibody. A647-SLAMF7 was conjugated with streptavidin (Abcam Streptavidin conjugation kit). 1 μL of Modifier Reagent was added to each 10 μL of antibody (A647-SLAMF7) to be labelled, and mixture was mixed gently. The cap from the vial of Streptavidin Conjugation Mix was removed, and the antibody sample (with added Streptavidin Conjugation Modifier reagent) was pipetted directly onto the lyophilized material. The mixture was resuspended gently by withdrawing and re-dispensing the liquid once or twice using a pipette. The cap was replaced onto the vial, and the vial was incubated for 3 hours in the dark at room temperature (20-25° C.). In some cases, conjugations were set up and incubated overnight. Longer incubation times had no negative effect on the conjugate.

After incubating, 1 μL of Streptavidin Conjugation Quencher reagent was add for every 10 μL of antibody used, and the mixture was mixed gently. The conjugate was available for use after 4 minutes. The conjugates did not require purification.

Herceptin was conjugated with biotin using an Abcam Biotin (Type A) Fast conjugation kit. 1 μL of Biotin (Type A) Modifier reagent was added to each 10 μL of Herceptin to be labelled, and the mixture was mixed gently. The cap was removed from the vial of Biotin (Type A) Conjugation Mix, and the antibody sample (with added Biotin (Type A) Modifier reagent) was pipetted directly onto the lyophilized material. It was resuspended gently by withdrawing and re-dispensing the liquid once or twice using a pipette. The cap was replaced on the vial, and the vial was incubated for 15 minutes at room temperature (20-25° C.). Longer incubation times had no negative effect on the conjugate. After incubating for 15 minutes, 1 μL of Biotin (Type A) Quencher reagent was added for every 10 μL of antibody used, and the mixture was mixed gently. The conjugate was available for use after 4 minutes. The conjugates did not require purification.

To conjugate the streptavidin and biotin, SLAMF7-streptavidin was mixed with Herceptin-biotin 1:1 in PBS and incubated for 1 hour at room temperature.

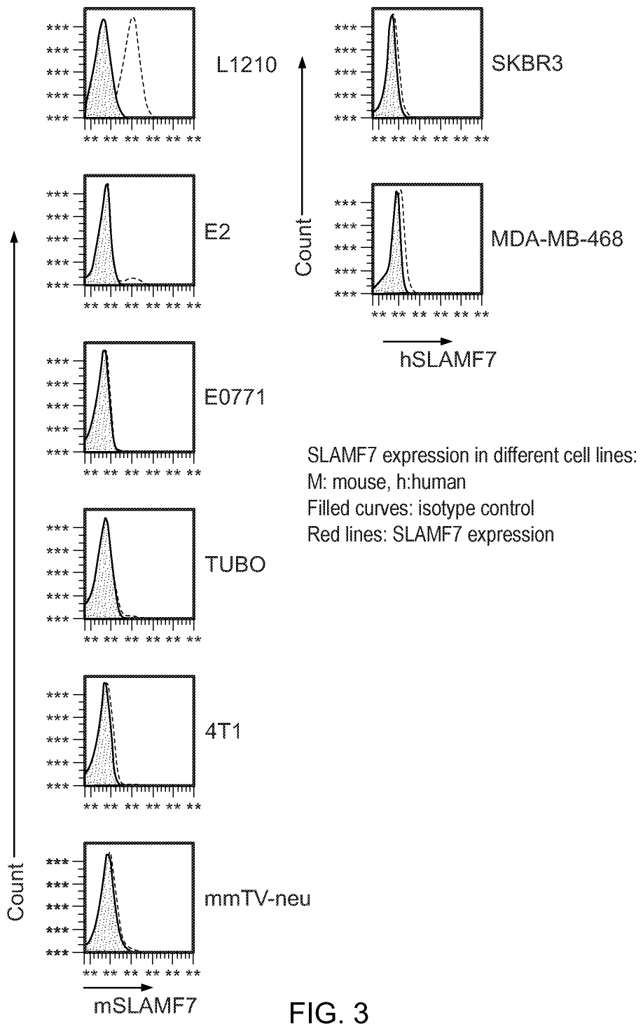

Eight cell lines were tested for SLAMF7 expression. Lymphocytic leukemia cell line L1210 cell line exhibited SLAMF7 expression, and other tested cell lines did not exhibit significant SLAMF7 expression (FIG. 3).

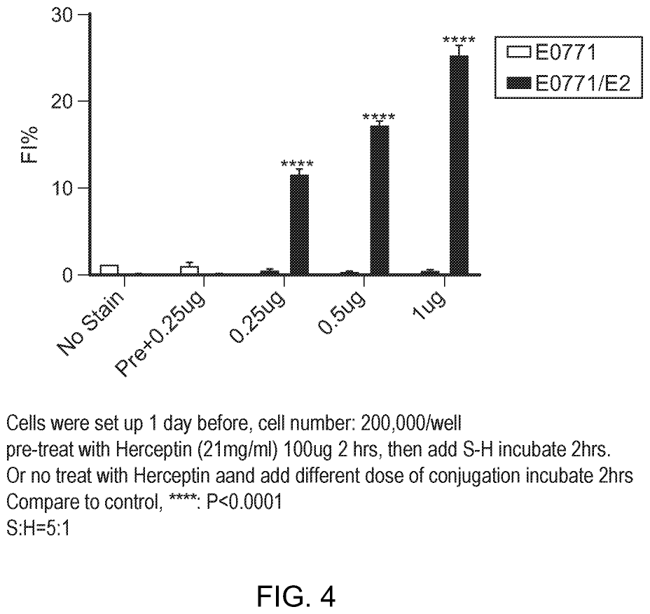

To test the ability of the S-H conjugate to bind to cells expressing human her2 (human epidermal growth factor receptor 2, a membrane protein overexpressed in 20% of human breast cancers), the following was performed. Briefly, the S-H conjugate was added to the cultured cell with (E0771/E2) or without human Her2 expression (E0771). The results demonstrated that only human Her2 expressing cells (E0771/E2) exhibited binding to the S-H conjugate. This binding affinity was blocked by pretreating the cells with Herceptin. These results demonstrate that the S-H conjugate can bind to cells expressing human Her2 (FIG. 4).

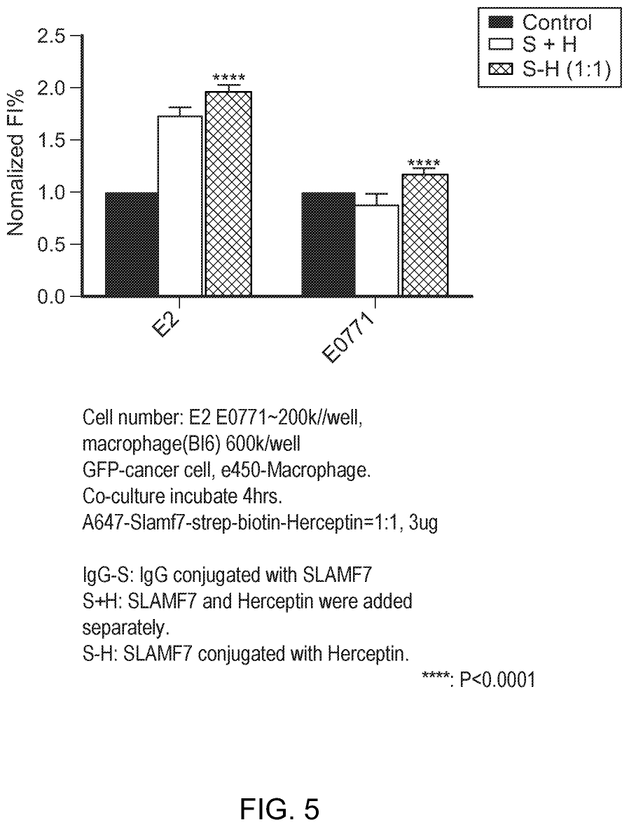

The following was performed to test that ability of the S-H conjugate to promote phagocytosis of SLAMF7+cells by macrophages. Briefly, phagocytosis was measured by co-culturing eFluor 450-labeled macrophages and CFSE-labeled tumor cells as follows. Tumor cells were plated in 12-well plates at 2×10⁵ cells/well and allowed to adhere for 2 hours. Then, 6×10⁵ macrophages (bone marrow-derived macrophages from C57BL/6 mice were added and co-cultured with the cancer cells for 4 hours at 37° C. with the different treatments. As shown in FIG. 5, the enhanced cancer-cell phagocytosis induced by S-H treatment was highly dependent on the Her2 expression level, as an elevated phagocytic activity of macrophages was not observed in cells that do not express Her2 (E0771). In addition, adding the S-H conjugate resulted in more phagocytosis than adding SLAMF7 and Herceptin separately. These results demonstrate that the S-H conjugate promotes the receptor-targeted phagocytosis of murine Her2 expression breast cancer cell lines.

The following was performed to test that ability of the S-H conjugate to promote phagocytosis of Her2+cells by macrophages in a manner that allows the macrophages to present antigens from the phagocytosed cells. Briefly, cOVA-expressing tumor cells were co-cultured with CFSE labelled macrophages for 2 days with the S-H. Cells were stained with APC-labelled anti-SIINFEK6/H-2Kb.

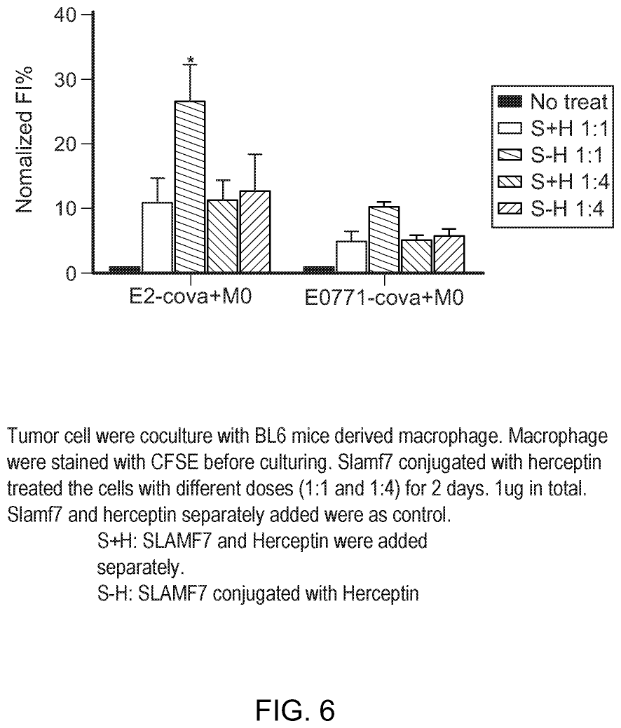

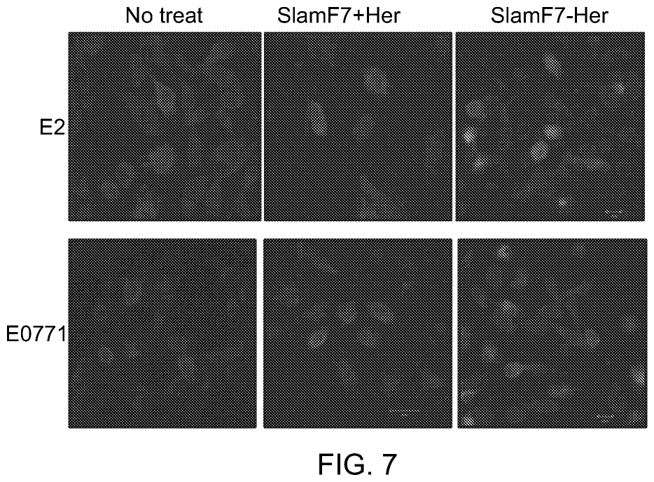

Fluorescent intensity was measured as the percentage of APC⁺ cells within CFSE labelled macrophages. As shown in FIG. 6, S-H treatment enhanced tumor antigen presentation in a Her2 receptor-targeted manner. FIG. 7 is an immunofluorescent representation of SIINFEKL/H-2Kb (pink), macrophages (blue), and macrophage cells positively labeled with SIINFEKL/H-2Kb. Compared with unconjugated SLAMF7 and Herceptin, the S-H conjugate enhanced the phagocytosis of cancer cells by macrophages and resulted in a significantly higher cross-presentation of ovalbumin peptide onto the major histocompatibility protein I complex, as demonstrated by staining of the SIINFEKL-H2kb complex on the macrophage surface. These results demonstrate that SLAMF7 mediated phagocytosis can promote antigen processing and immune activation of professional APCs and that S-H treatment can enhance the tumor antigen presentation in a Her2-targeted manner.

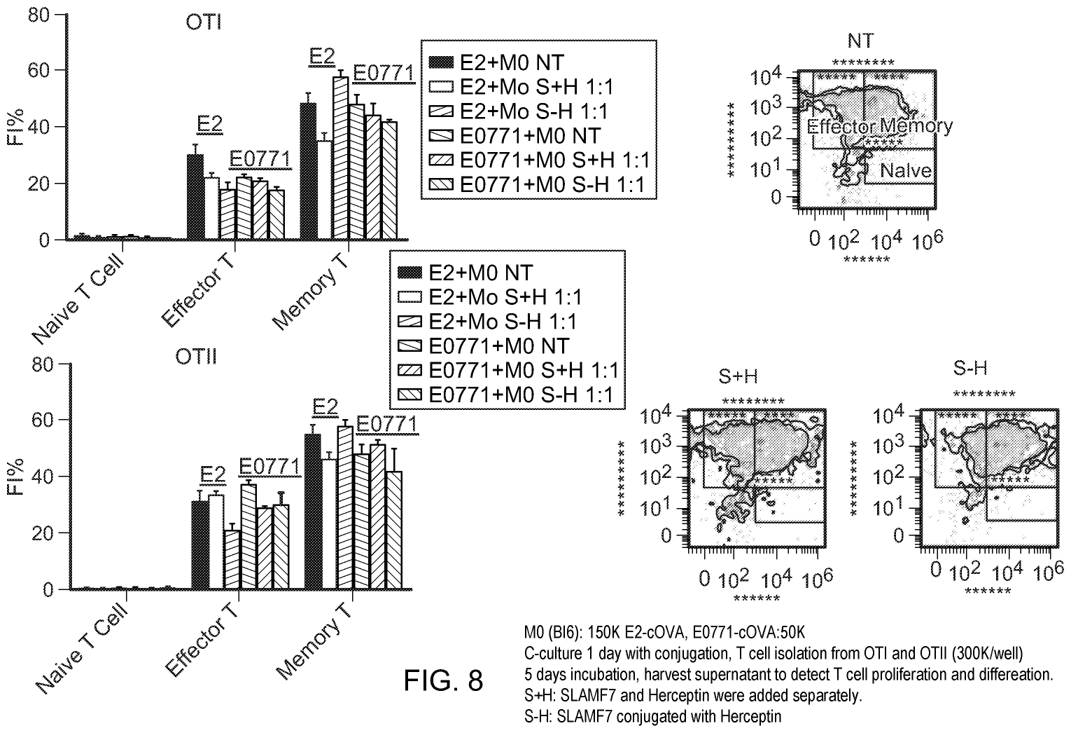

The following was performed to test that ability of the S-H conjugate to promote phagocytosis of Her2⁺ cells by macrophages in a manner that results in the stimulation of T cells activation. Briefly, OVA-specific CD4⁺ and CD8⁺ T cells were first isolated from transgenic OT-II and OT-I mice, respectively. The T cells were then added to a macrophage-cancer cell co-culture previously treated with the S-H conjugate, a non-conjugate of SLAMF7, and a non-conjugate of Herceptin. As shown in FIG. 8, treatment with S-H resulted in an increase in CD44^{high}CD62L^{high} memory T cell in both CD4⁺ and CD8⁺ T cell populations. These results demonstrate that the S-H conjugate has the ability to promote the Her2-targeted phagocytic clearance of cancer cells and to activate downstream adaptive immune responses.

### Example 2

### In Vivo Confirmation of Anti-Cancer Activity

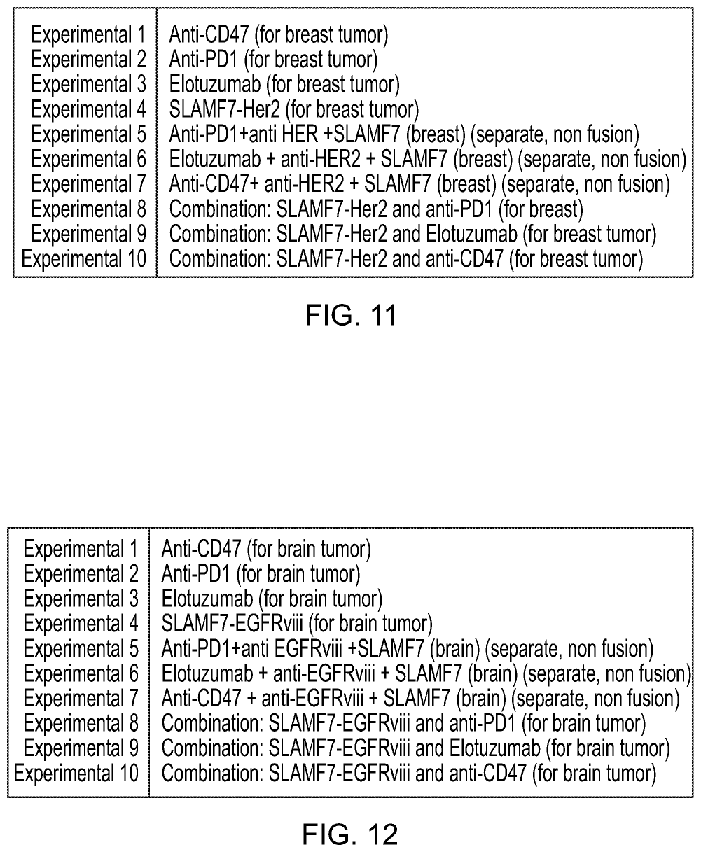

In vivo experiments are performed using tumor-specific (e.g., breast or brain) mouse models. In preparation for the in vivo experiments, a fusion polypeptide (or polypeptide conjugate) having a tumor-targeting moiety (e.g., an antibody having binding affinity for an antigen expressed by cancer cells within a mammal such as a Herceptin antibody or and anti-EGFR antibody) and a phagocyte engaging moiety (e.g., SLAMF7) is synthesized and characterized. Affinity to tumor cells is tested, followed by immune cell activation, testing for both innate and adaptive immunity. The fusion polypeptides are administered to mice. Several experimental groups of mice to provide control groups, as well as test combination therapies, are used as outlined in FIG. 11 for breast cancer and FIG. 12 for brain cancer. Delivery and additional properties are characterized and imaged. Pre-clinical efficacy is confirmed by measuring anti-tumor activity and/or survival.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a first molecule having said ability to bind to cancer cells and a second molecule having said ability to bind to antigen presenting cells, wherein said first molecule is non-covalently linked to said second molecule, wherein incubation of said cancer cells and said antigen presenting cells in the presence of said composition results in increased phagocytosis of said cancer cells by said antigen presenting cells as compared to said level of phagocytosis of comparable cancer cells by comparable antigen presenting cells in the absence of said composition.

2. The composition of claim 1, wherein said first molecule comprises a streptavidin sequence, wherein said second molecule comprises a biotin sequence, and wherein said first molecule is non-covalently linked to said second molecule via a non-covalent linkage between said streptavidin sequence and said biotin sequence.

3. The composition of claim 1, wherein said second molecule comprises a streptavidin sequence, wherein said first molecule comprises a biotin sequence, and wherein said first molecule is non-covalently linked to said second molecule via a non-covalent linkage between said streptavidin sequence and said biotin sequence.

4-5. (canceled)

6. The composition of claim 1, wherein said molecule having said ability to bind to antigen presenting cells is a polypeptide.

7. The composition of claim 6, wherein said polypeptide comprises a SLAMF7 amino acid sequence.

8-10. (canceled)

11. The composition of claim 1, wherein said antigen presenting cells are macrophages.

12. The composition of claim 1, wherein said antigen presenting cells are human macrophages.

13. The composition of claim 1, wherein said increased phagocytosis is at least a two-fold increase.

14. The composition of claim 1, wherein said increased phagocytosis is at least a four-fold increase.

15. A fusion polypeptide comprising a first amino acid segment having the ability to bind to cancer cells and a second amino acid segment having the ability to bind to antigen presenting cells, wherein incubation of said cancer cells and said antigen presenting cells in the presence of said fusion polypeptide results in increased phagocytosis of said cancer cells by said antigen presenting cells as compared to the level of phagocytosis of comparable cancer cells by comparable antigen presenting cells in the absence of said fusion polypeptide.

16. The fusion polypeptide of claim 15, wherein said first amino acid segment is linked to said second amino acid segment via a linker amino acid sequence.

17-18. (canceled)

19. The fusion polypeptide of claim 15, wherein said second amino acid segment comprises a SLAMF7 amino acid sequence.

20-22. (canceled)

23. The fusion polypeptide of claim 15, wherein said antigen presenting cells are macrophages.

24. The fusion polypeptide of claim 15, wherein said antigen presenting cells are human macrophages.

25. The fusion polypeptide of claim 15, wherein said increased phagocytosis is at least a two-fold increase.

26. The fusion polypeptide of claim 15, wherein said increased phagocytosis is at least a four-fold increase.

27. A method for treating a mammal having cancer, said method comprising administering to said mammal a composition or a fusion polypeptide,
wherein said composition comprises a first molecule having an ability to bind to cancer cells and a second molecule having an ability to bind to antigen presenting cells, wherein said first molecule is non-covalently linked to said second molecule, wherein incubation of said cancer cells and said antigen presenting cells in the presence of said composition results in increased phagocytosis of said cancer cells by said antigen presenting cells as compared to said level of phagocytosis of comparable cancer cells by comparable antigen presenting cells in the absence of said composition, and
wherein said fusion polypeptide comprises a first amino acid segment having an ability to bind to cancer cells and a second amino acid segment having an ability to bind to antigen presenting cells, wherein incubation of said cancer cells and said antigen presenting cells in the presence of said fusion polypeptide results in increased phagocytosis of said cancer cells by said antigen presenting cells as compared to the level of phagocytosis of comparable cancer cells by comparable antigen presenting cells in the absence of said fusion polypeptide.

28. The method of claim 27, wherein said mammal is a human.

29. The method of claim 27, wherein said cancer is breast cancer, brain cancer, prostate cancer, lung cancer, or colorectal cancer.

30. The method of claim 27, wherein said composition is administered by injection, ingestion, or inhalation.


SEQUENCE LISTING (16 sequences)

SEQ ID NO: 1 - protein, 202 aa, Mus musculus
SGTLKKVAGALDGSVTFTLNITEIKVDYVVWTFNTFFLAMVKKDGVTSQSSNKERIVFPDGLYSMKLSQLKKNDSGAYRAEIYSTSSQASLIQEYVLHVYKHLSRPKVTIDRQSNKNGTCVINLTCSTDQDGENVTYSWKAVGQGDNQFHDGATLSIAWRSGEKDQALTCMARNPVSNSFSTPVFPQKLCEDAATDLTSLRG

SEQ ID NO: 2 - protein, 449 aa, Humanize Mouse
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 4 - protein, 313 aa, Homo sapiens
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 5 - protein, 204 aa, Homo sapiens
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 6 - protein, 102 aa, Homo sapiens
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHV

SEQ ID NO: 7 - protein, 76 aa, Homo sapiens
PKVTMGLQSNKNGTCVTNLTCCMEHGEEDVIYTWKALGQAANESHNGSILPISWRWGESDMTFICVARNPVSRNFS

SEQ ID NO: 8 - protein, 95 aa, Homo sapiens
GSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVY

SEQ ID NO: 9 - protein, 78 aa, Homo sapiens
KELVGSVGGAVTFPLKSKVKQVDSIVWTFNTTPLVTIQPEGGTIIVTQNRNRERVDFPDGGYSLKLSKLKKNDSGIYY

SEQ ID NO: 10 - protein, 333 aa, Mus musculus
MARFSTYIIFTSVLCQLTVTAASGTLKKVAGALDGSVTFTLNITEIKVDYVVWTFNTFFLAMVKKDGVTSQSSNKERIVFPDGLYSMKLSQLKKNDSGAYRAEIYSTSSQASLIQEYVLHVYKHLSRPKVTIDRQSNKNGTCVINLTCSTDQDGENVTYSWKAVGQGDNQFHDGATLSIAWRSGEKDQALTCMARNPVSNSFSTPVFPQKLCEDAATDLTSLRGILYILCFSAVLILFAVLLTIFHTTWIKKGKGCEEDKKRVDRHQEMPDLCPHLEENADYDTIPYTEKRRPEEDAPNTFYSTVQIPKVVKSPSSLPAKPLVPRSLSFENVI

SEQ ID NO: 11 - protein, 311 aa, Mus musculus
SGTLKKVAGALDGSVTFTLNITEIKVDYVVWTFNTFFLAMVKKDGVTSQSSNKERIVFPDGLYSMKLSQLKKNDSGAYRAEIYSTSSQASLIQEYVLHVYKHLSRPKVTIDRQSNKNGTCVINLTCSTDQDGENVTYSWKAVGQGDNQFHDGATLSIAWRSGEKDQALTCMARNPVSNSFSTPVFPQKLCEDAATDLTSLRGILYILCFSAVLILFAVLLTIFHTTWIKKGKGCEEDKKRVDRHQEMPDLCPHLEENADYDTIPYTEKRRPEEDAPNTFYSTVQIPKVVKSPSSLPAKPLVPRSLSFENVI

SEQ ID NO: 12 - protein, 86 aa, Mus musculus
KKVAGALDGSVTFTLNITEIKVDYVVWTFNTFFLAMVKKDGVTSQSSNKERIVFPDGLYSMKLSQLKKNDSGAYRAEIYSTSSQAS

SEQ ID NO: 13 - protein, 76 aa, Mus musculus
PKVTIDRQSNKNGTCVINLTCSTDQDGENVTYSWKAVGQGDNQFHDGATLSIAWRSGEKDQALTCMARNPVSNSFS

SEQ ID NO: 14 - protein, 92 aa, Mus musculus
GALDGSVTFTLNITEIKVDYVVWTFNTFFLAMVKKDGVTSQSSNKERIVFPDGLYSMKLSQLKKNDSGAYRAEIYSTSSQASLIQEYVLHVY

SEQ ID NO: 15 - protein, 70 aa, Mus musculus
RPKVTIDRQSNKNGTCVINLTCSTDQDGENVTYSWKAVGQGDNQFHDGATLSIAWRSGEKDQALTCMARN

SEQ ID NO: 16 - protein, 214 aa, Humanize Mouse
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC